(19) United States
(12) Patent Application Publication
(10) Pub. No.: US 2021/0085233 A1
(43) Pub. Date: Mar. 25, 2021

(54) Wearable Device for Determining and Monitoring Emotional States of a User, and a System Thereof

(71) Applicant: Monsoon Design Studios LLC, Los Gatos, CA (US)

(72) Inventors: Giuliana Kotikela, Los Gatos, CA (US); Aditya Sane, San Jose, CA (US); Daniel Housman, Newton, MA (US); Purav Gandhi, Ahmedabad (IN); Anshu Chittora, Ahmedabad (IN); Sanandan Sudhir, Ahmedabad (IN); Rohan Vinodkumar Sharma, Ahmedabad (IN); Tarkas Pavankumar Vinodkumar, Ahmedabad (IN); Upendra Patel, Ahmedabad (IN)

(21) Appl. No.: 17/030,752

(22) Filed: Sep. 24, 2020

(30) Foreign Application Priority Data
Sep. 24, 2019 (IN) 201941038609

(51) Int. Cl. A61B 5/16; A61B 5/00; A61B 5/11; A61B 5/0205; A61B 5/1455; A61B 5/053

(52) U.S. Cl. CPC A61B 5/165; A61B 5/6802; A61B 5/742; A61B 5/1126; A61B 5/0205; A61B 5/14552; A61B 5/02416; A61B 5/4266; A61B 5/4803; A61B 5/0002; A61B 5/7264; A61B 2560/0214; A61B 2562/0219; A61B 5/0533

(57) ABSTRACT

The present disclosure provides a wearable device, which includes: a frame adapted to be worn on a body part of a user. The frame includes: one or more sensors configured in a housing and operative to sense one or more parameters associated with the body of the user to generate one or more signals indicative of the one or more sensed parameters when the wearable device is worn; a processor configured in the housing and operatively coupled with the one or more sensors, the processor operative to analyze the one or more signals to extract one or more attributes associated with the user from the one or more signals, wherein the one or more attributes being indicative of emotional states of the user; and a display device configured at a portion of the housing and operatively coupled with the processor, wherein the display device is configured to indicate, based on the extracted one or more attributes of the user, the emotional states of the user.

### TECHNICAL FIELD

The present disclosure relates to the field of wearable smart devices. In particular, the present disclosure relates to a wearable device for determining and monitoring emotional states of a user.

### BACKGROUND

The background description includes information that may be useful in understanding the present invention. It is not an admission that any of the information provided herein is prior art or relevant to the presently claimed invention, or that any publication specifically or implicitly referenced is prior art.

The market for wearable smart devices has significantly grown in the 21st century, because of their small and compact structure, and capability to provide many applications for wearer. These wearable smart devices are improving ease of access, quality of life, and help monitor bio-physiological activity within the wearer's body.

People are often experiencing various mental health related issues due to work pressure, family, environment, and other factors. This directly affects the overall health and wellbeing of people. Wearable smart devices may facilitate health and emotional monitoring and influence wellbeing of users.

Various wearable health monitoring devices are available in the market to monitor and determine the health status of users, e.g., calories burned by the users, number of steps covered by the users, and pulse of the user, however, current devices on the market do not have the ability to calculate the wearer's emotional states.

There is therefore a need in the art to provide a system and device that can determine and monitor emotional states or moods of a user in real-time with minimal user participation.

### OBJECTS OF THE DISCLOSURE

A general object of the present disclosure is to provide a wearable device for a user.

Another object of the present disclosure is to provide a wearable device for a user to determine emotional states of a user.

Another object of the present disclosure is to provide a wearable device which visually depicts emotional states of a user.

### SUMMARY

The present disclosure relates to the field of wearable smart devices. In particular, the present disclosure relates to a wearable device for determining and monitoring emotional states of a user.

In an aspect, the present disclosure provides a wearable device for determining emotional states of a user, which includes: a frame adapted to be worn on a body part of the user. The frame includes: one or more sensors configured in a housing and operative to sense one or more parameters associated with the body of the user to generate one or more signals indicative of the one or more sensed parameters when the wearable device is worn; a processor configured in the housing and operatively coupled with the one or more sensors, the processor configured to: analyze the one or more signals to extract one or more attributes associated with the user from the one or more signals, wherein said one or more attributes being indicative of emotional states of the user; and retrieve, from a database operatively coupled to the processor, recommended emotional states based on a deviation of the indicated emotional states of the user from a preferred emotional state of the user, wherein the recommended emotional states, when adopted by the user, facilitates the indicated emotional states of the user to tend towards the preferred emotional state of the user; and a display device configured at a portion of the housing and operatively coupled with the processor, wherein the display device is configured to indicate, based on the extracted one or more attributes of the user, any or a combination of the emotional states of the user and the corresponding recommended emotional states for the user.

In an embodiment, the device can be configured with a power storage device configured to supply power to the one or more sensors, the processor, and the display device.

In another embodiment, the device can be configured with a set of contacts adapted to receive an external power supply to charge the power storage device.

In another embodiment, the one or more sensors and the processor can be configured on a flexible printed circuit board configured in the housing of the device.

In another embodiment, the one or more sensors can be selected from a group including accelerometer, one or more sources of electromagnetic radiation, a photodetector, a galvanic skin response sensor, a microphone, a GPS unit, and any combination thereof.

In another embodiment, the one or more sensors can be the accelerometer, and wherein the processor can be configured to: receive, from the accelerometer, a set of signals associated with movement of the body part of the user. The processor can be configured to analyze the received one or more signals to extract one or more attributes associated with the body part of the user from the received one or more signals, wherein the one or more attributes are at least partially indicative of the emotional states of the user.

In another embodiment, the one or more sensors can be a combination of the one or more sources of electromagnetic radiation and the photodetector, and wherein the processor can be configured to: operate the one or more sources of electromagnetic radiation to emit electromagnetic radiation corresponding to one or more wavelengths, the electromagnetic radiation aimed towards the body part of the user; and receive, from the photodetector, one or more signals associated with interaction of the emitted electromagnetic radiation with the body part of the user, the interaction being any or a combination of transmittance, reflection and absorption. The processor can be configured to analyze the received one or more signals to extract one or more attributes associated with the body part of the user from the received one or more signals, the one or more attributes being indicative of any or a combination of pulse rate and oxygen saturation of the user, and wherein the one or more attributes are at least partially indicative of the emotional states of the user.

In another embodiment, the one or more sensors can be the galvanic skin response sensor, and wherein the processor can be configured to: receive, from the galvanic skin response sensor, one or more signals associated with electrical conductivity of the body part of the user. The processor can be configured to analyze the received one or more signals to extract one or more attributes associated with the body part of the user from the received one or more signals, the one or more attributes being indicative of quantity of sweat present on the body part of the user and temperature of the body part of the user, and wherein the one or more attributes are at least partially indicative of the emotional states of the user.

In another embodiment, the one or more sensors can be the microphone, and wherein the processor can be configured to: receive, from the microphone, one or more signals associated with speech of the user. The processor can be configured to analyze the received one or more signals to extract one or more attributes associated with the body part of the user from the received one or more signals, the one or more attributes pertaining to any or a combination of pitch, timbre, volume, speed and tone, and wherein the one or more attributes are at least partially indicative of the emotional states of the user.

In another embodiment, the processor can be configured to retrieve the recommended emotional states for the user from a database operatively coupled to it.

In another embodiment, the display device can be configured to emit a first light of a predefined color out of a set of colors, the predefined color being indicative of the emotional states of the user, and wherein the predefined color is selected based on a predefined association of the set of colors with different emotional states.

In another embodiment, the display device can be configured to emit a second light of a predefined color out of a set of colors, the predefined color being indicative of the recommended emotional states for the user, and wherein the predefined color is selected based on a predefined association of the set of colors with different emotional states.

In another embodiment, the display device can include a light source coupled with a diffuser, the light source adapted to emit light of different colors and the diffuser adapted to provide a soft illumination of the display device.

In another embodiment, the device can include a memory unit configured to store information pertaining to a log of operations of the device, the log of operations including any or a combination of the one or more signals from the one or more sensors, the extracted one or more attributes, the emotional states of the user, the corresponding retrieved recommended emotional states for the user, and a time stamp for the log of operations.

In another embodiment, the device includes a transceiver unit operatively coupled with the processor, the transceiver unit configured to communicatively couple the device with an external mobile device, the communicative coupling being any of wireless and wired.

In another embodiment, the external mobile device can be configured with an application executable to communicatively couple with the wearable device.

In another embodiment, the communicative coupling can be a wireless communicative coupling, being through any or a combination of Wi-Fi, Bluetooth, radio, and mobile network. In an exemplary embodiment, the communicative coupling is through Bluetooth.

In another embodiment, the external mobile device can be a remote server, the server operatively coupled with a computing device, the computing device including a processor coupled with a memory, the memory storing instructions executable by the processor to perform one or more analyses of the log of operations of the device.

In an aspect, the present disclosure provides a system to determine emotional states of a user using the wearable device. The system includes a processor operatively coupled with a memory, the memory storing instructions executable by the processor to: receive, from one or more sensors configured in the device operatively coupled to it, one or more signals corresponding to one or more sensed body parameters of the body of the user on which the wearable device is worn, wherein the processor is operative to analyze the one or more signals to extract one or more attributes associated with the user from the one or more signals, said one or more attributes being indicative of emotional states of the user; retrieve, from a database operatively coupled to the processor, recommended emotional states based on a deviation of the indicated emotional states of the user from a preferred emotional state of the user, wherein the recommended emotional states, when adopted by the user, facilitates the indicated emotional states of the user to tend towards the preferred emotional state of the user; and a display device configured at a portion of the housing and operatively coupled with the processor, wherein the display device is configured to indicate, based on the extracted one or more attributes of the user, any or a combination of the emotional states of the user and the corresponding recommended emotional states for the user.

In an embodiment, the processor can be operatively coupled with a learning engine, the learning engine configured to determine the emotional states of the user from one or more analyses performed on the extracted one or more attributes of the user.

In another embodiment, the learning engine can be trained to determine the emotional states of the user based on historical data comprising the one or more attributes and the corresponding emotional states of the user.

In another embodiment, the learning engine can be trained to determine the emotional states of the user based on simulated historical data comprising one or more attributes and the corresponding emotional states.

In another embodiment, the learning engine can be configured with a computer implementable machine learning model comprising a classifier model, a generalized regression and non-linear regression. In another embodiment, the classifier model can include any or a combination of a support vector machine (SVM), a Gaussian Mixture model (GMM), a k-nearest neighbor classifier, and a neural network classifier.

In another embodiment, the system can include one or more wearable devices associated with any of one or more users, the system configured to depict any or a combination of emotional states of any of the one or more users and corresponding recommended emotional states for any of the one or more users.

In an aspect, the present disclosure provides a method to determine emotional states of a user using the wearable device. The method includes: receiving, at a processor from one or more sensors configured in the device operatively coupled to it, one or more signals corresponding to one or more sensed body parameters of the body of the user on which the wearable device is worn, wherein the processor is operative to analyze the one or more signals to extract one or more attributes associated with the user from the one or more signals, said one or more attributes being indicative of emotional states of the user; retrieving, at the processor from a database operatively coupled to the processor, recommended emotional states based on a deviation of the indicated emotional states of the user and a preferred emotional state of the user, wherein the recommended emotional states, when adopted by the user, facilitates the indicated emotional states of the user to tend towards the preferred emotional state of the user; and depicting, on a display device operatively coupled to the processor, based on the extracted one or more attributes of the user, any or a combination of the emotional states of the user and the corresponding recommended emotional states for the user.

While the invention has been described by way of example and in terms of the specific embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present invention and are incorporated in and constitute a part of this specification. The drawings illustrate exemplary embodiments of the present invention and, together with the description, serve to explain the principles of the present invention.

FIGS. 1A and 1B illustrate exemplary representations of a wearable device for determining emotional states of a user, in accordance with an embodiment of the present disclosure.

FIG. 1C illustrates an exploded view of a wearable device for determining emotional states of a user, in accordance with an embodiment of the present disclosure.

FIGS. 2A-2C illustrate exemplary representations of a charger for the proposed wearable device, in accordance with an embodiment of the present disclosure.

FIG. 3 illustrates an exemplary representation of the flexible printed circuit board of the wearable device.

FIG. 4 illustrates an exemplary block diagram for a system implementable on the proposed wearable device to determine emotional states of the user, in accordance with an embodiment of the present disclosure.

FIG. 5 illustrates exemplary system architecture of the proposed wearable device, in accordance with an embodiment of the present disclosure.

FIG. 6 illustrates exemplary network architecture in which or with which proposed system can be implemented, in accordance with an embodiment of the present disclosure.

FIG. 7 illustrates an exemplary flow diagram for a method implementable on the proposed wearable device to determine emotional states of the user, in accordance with an embodiment of the present disclosure.

FIG. 8 illustrates an exemplary computer system in which or with which embodiments of the present invention can be utilized in accordance with embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Exemplary embodiments will now be described more fully hereinafter with reference to the accompanying drawings, in which exemplary embodiments are shown. These exemplary embodiments are provided only for illustrative purposes and so that this disclosure will be thorough and complete and will fully convey the scope of the invention to those of ordinary skill in the art. The invention disclosed may, however, be embodied in many different forms and should not be construed as limited to the embodiments set forth herein. Various modifications will be readily apparent to persons skilled in the art. The general principles defined herein may be applied to other embodiments and applications without departing from the spirit and scope of the invention. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Also, the terminology and phraseology used is for the purpose of describing exemplary embodiments and should not be considered limiting. Thus, the present invention is to be accorded the widest scope encompassing numerous alternatives, modifications, and equivalents consistent with the principles and features disclosed. For purpose of clarity, details relating to technical material that is known in the technical fields related to the invention have not been described in detail so as not to unnecessarily obscure the present invention.

The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The present disclosure relates to the field of wearable smart devices. In particular, the present disclosure relates to a wearable device for determining and monitoring emotional states of a user.

In an aspect, the present disclosure provides a wearable device for determining emotional states of a user, which includes: a frame adapted to be worn on a body part of the user. The frame includes: one or more sensors configured in a housing and operative to sense one or more parameters associated with the body of the user to generate one or more signals indicative of the one or more sensed parameters when the wearable device is worn; a processor configured in the housing and operatively coupled with the one or more sensors, the processor configured to: analyze the one or more signals to extract one or more attributes associated with the user from the one or more signals, wherein said one or more attributes being indicative of emotional states of the user; and retrieve, from a database operatively coupled to the processor, recommended emotional states based on a deviation of the indicated emotional states of the user from a preferred emotional state of the user, wherein the recommended emotional states, when adopted by the user, facilitates the indicated emotional states of the user to tend towards the preferred emotional state of the user; and a display device configured at a portion of the housing and operatively coupled with the processor, wherein the display device is configured to indicate, based on the extracted one or more attributes of the user, any or a combination of the emotional states of the user and the corresponding recommended emotional states for the user.

In an embodiment, the device can be configured with a power storage device configured to supply power to the one or more sensors, the processor, and the display device.

In another embodiment, the device can be configured with a set of contacts adapted to receive an external power supply to charge the power storage device.

In another embodiment, the one or more sensors and the processor can be configured on a flexible printed circuit board configured in the housing of the device.

In another embodiment, the one or more sensors can be selected from a group including accelerometer, one or more sources of electromagnetic radiation, a photodetector, a galvanic skin response sensor, a microphone, a GPS unit, and any combination thereof.

In another embodiment, the one or more sensors can be the accelerometer, and wherein the processor can be configured to: receive, from the accelerometer, a set of signals associated with movement of the body part of the user. The processor can be configured to analyze the received one or more signals to extract one or more attributes associated with the body part of the user from the received one or more signals, wherein the one or more attributes are at least partially indicative of the emotional states of the user.

In another embodiment, the one or more sensors can be a combination of the one or more sources of electromagnetic radiation and the photodetector, and wherein the processor can be configured to: operate the one or more sources of electromagnetic radiation to emit electromagnetic radiation corresponding to one or more wavelengths, the electromagnetic radiation aimed towards the body part of the user; and receive, from the photodetector, one or more signals associated with interaction of the emitted electromagnetic radiation with the body part of the user, the interaction being any or a combination of transmittance, reflection and absorption. The processor can be configured to analyze the received one or more signals to extract one or more attributes associated with the body part of the user from the received one or more signals, the one or more attributes being indicative of any or a combination of pulse rate and oxygen saturation of the user, and wherein the one or more attributes are at least partially indicative of the emotional states of the user.

In another embodiment, the one or more sensors can be the galvanic skin response sensor, and wherein the processor can be configured to: receive, from the galvanic skin response sensor, one or more signals associated with electrical conductivity of the body part of the user. The processor can be configured to analyze the received one or more signals to extract one or more attributes associated with the body part of the user from the received one or more signals, the one or more attributes being indicative of quantity of sweat present on the body part of the user and temperature of the body part of the user, and wherein the one or more attributes are at least partially indicative of the emotional states of the user.

In another embodiment, the one or more sensors can be the microphone, and wherein the processor can be configured to: receive, from the microphone, one or more signals associated with speech of the user. The processor can be configured to analyze the received one or more signals to extract one or more attributes associated with the body part of the user from the received one or more signals, the one or more attributes pertaining to any or a combination of pitch, timbre, volume, speed and tone, and wherein the one or more attributes are at least partially indicative of the emotional states of the user.

In another embodiment, the processor can be configured to retrieve the recommended emotional states for the user from a database operatively coupled to it.

In another embodiment, the display device can be configured to emit a first light of a predefined color out of a set of colors, the predefined color being indicative of the emotional states of the user, and wherein the predefined color is selected based on a predefined association of the set of colors with different emotional states.

In another embodiment, the display device can be configured to emit a second light of a predefined color out of a set of colors, the predefined color being indicative of the recommended emotional states for the user, and wherein the predefined color is selected based on a predefined association of the set of colors with different emotional states.

In another embodiment, the display device can include a light source coupled with a diffuser, the light source adapted to emit light of different colors and the diffuser adapted to provide a soft illumination of the display device.

In another embodiment, the device can include a memory unit configured to store information pertaining to a log of operations of the device, the log of operations including any or a combination of the one or more signals from the one or more sensors, the extracted one or more attributes, the emotional states of the user, the corresponding retrieved recommended emotional states for the user, and a time stamp for the log of operations.

In another embodiment, the device includes a transceiver unit operatively coupled with the processor, the transceiver unit configured to communicatively couple the device with an external mobile device, the communicative coupling being any of wireless and wired.

In another embodiment, the external mobile device can be configured with an application executable to communicatively couple with the wearable device.

In another embodiment, the communicative coupling can be a wireless communicative coupling, being through any or a combination of Wi-Fi, Bluetooth, radio, and mobile network. In an exemplary embodiment, the communicative coupling is through Bluetooth.

In another embodiment, the external mobile device can be a remote server, the server operatively coupled with a computing device, the computing device including a processor coupled with a memory, the memory storing instructions executable by the processor to perform one or more analyses of the log of operations of the device.

In an aspect, the present disclosure provides a system to determine emotional states of a user using the wearable device. The system includes a processor operatively coupled with a memory, the memory storing instructions executable by the processor to: receive, from one or more sensors configured in the device operatively coupled to it, one or more signals corresponding to one or more sensed body parameters of the body of the user on which the wearable device is worn, wherein the processor is operative to analyze the one or more signals to extract one or more attributes associated with the user from the one or more signals, said one or more attributes being indicative of emotional states of the user; retrieve, from a database operatively coupled to the processor, recommended emotional states based on a deviation of the indicated emotional states of the user and a preferred emotional state of the user, wherein the recommended emotional states, when adopted by the user, facilitates the indicated emotional states of the user to tend towards the preferred emotional state of the user; and a display device configured at a portion of the housing and operatively coupled with the processor, wherein the display device is configured to indicate, based on the extracted one or more attributes of the user, any or a combination of the emotional states of the user and the corresponding recommended emotional states for the user.

In an embodiment, the processor can be operatively coupled with a learning engine, the learning engine configured to determine the emotional states of the user from one or more analyses performed on the extracted one or more attributes of the user.

In another embodiment, the learning engine can be trained to determine the emotional states of the user based on historical data comprising the one or more attributes and the corresponding emotional states of the user.

In another embodiment, the learning engine can be trained to determine the emotional states of the user based on simulated historical data comprising one or more attributes and the corresponding emotional states.

In another embodiment, the learning engine can be configured with a computer implementable machine learning model comprising a classifier model, a generalized regression and non-linear regression. In another embodiment, the classifier model can include any or a combination of a support vector machine (SVM), a Gaussian Mixture model (GMM), a k-nearest neighbor classifier, and a neural network classifier.

In another embodiment, the system can include one or more wearable devices associated with any of one or more users, the system configured to depict any or a combination of emotional states of any of the one or more users and corresponding recommended emotional states for any of the one or more users.

In an aspect, the present disclosure provides a method to determine emotional states of a user using the wearable device. The method includes: receiving, at a processor from one or more sensors configured in the device operatively coupled to it, one or more signals corresponding to one or more sensed body parameters of the body of the user on which the wearable device is worn, wherein the processor is operative to analyze the one or more signals to extract one or more attributes associated with the user from the one or more signals, said one or more attributes being indicative of emotional states of the user; retrieving, at the processor from a database operatively coupled to the processor, recommended emotional states based on a deviation of the indicated emotional states of the user and a preferred emotional state of the user, wherein the recommended emotional states, when adopted by the user, facilitates the indicated emotional states of the user to tend towards the preferred emotional state of the user; and depicting, on a display device operatively coupled to the processor, based on the extracted one or more attributes of the user, any or a combination of the emotional states of the user and the corresponding recommended emotional states for the user.

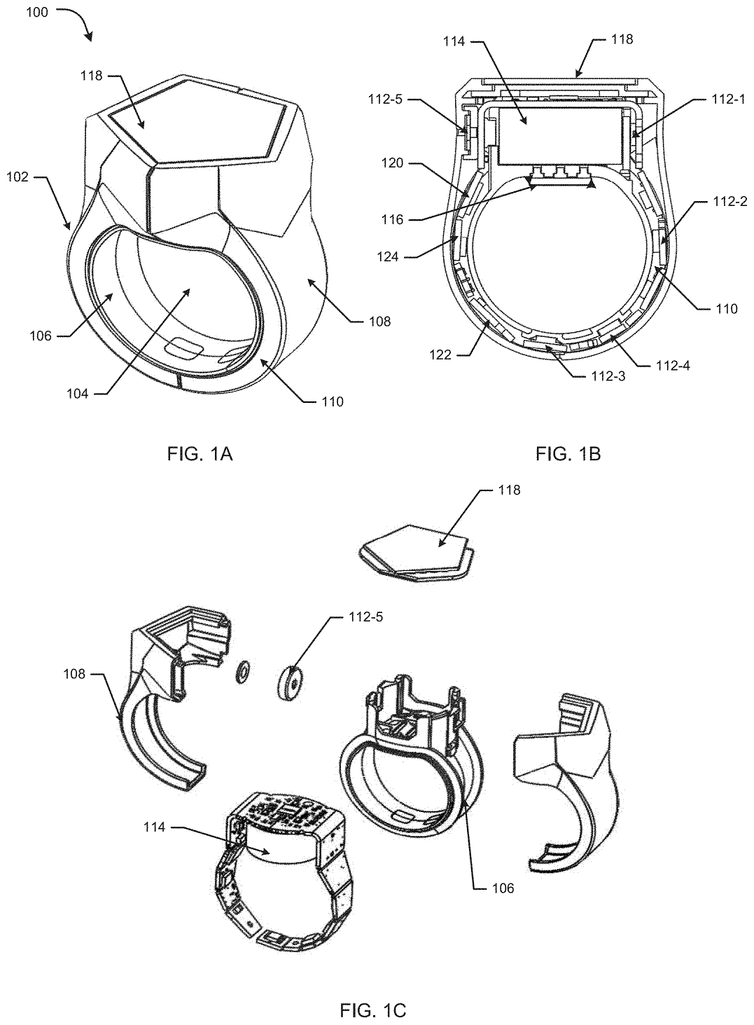

FIGS. 1A and 1B illustrate exemplary representations of a wearable device for determining emotional states of a user, in accordance with an embodiment of the present disclosure.

FIG. 1C illustrates an exploded view of a wearable device for determining emotional states of a user, in accordance with an embodiment of the present disclosure.

The wearable device 100 (herein, also referred to as “device”) includes a frame 102 that is adapted to be worn on a body part of a user.

In an exemplary embodiment, the frame 102 can include a slot 104 of a cross-section that can be adapted to fit around the body part. The frame 102 can be adapted to be worn around a body part such as a finger or wrist, and therefore any form factor can be configured for the proposed device depending on which body part the user wishes to put on the device 100 on/at, all of which form factors/possible configurations are well within the scope of the present disclosure.

In another embodiment, the frame 102 can include an inner section 106 that is adapted to be fit around the configured/desired body part. The inner section 106 can have a similar cross-section as the slot 104 and can include a cavity in on its outer surface. The frame 102 can have an outer section 108 adapted to fit over the inner section 106 such that the outer section 108 and the cavity of the inner section 106 form an enclosure or a housing 110.

In an exemplary embodiment, the inner section 104 can be made of a material such as plastic, and the outer section 106 can be made of a material such as steel, carbon fiber and titanium alloy. The outer section 106 is made of stronger materials in order that it can sustain greater wear, as the outer section 106 is exposed to the external environment. In another exemplary embodiment, a sealing element such as rubber or silicone can be provided between the inner section 104 and the outer section 106 so that the housing 110 is protected from moisture and dust from the external environment.

In another embodiment, one or more sensors (112-1, 112-2 . . . 112-n; herein, individually and collectively designated 112) are configured in the housing, and are adapted to sense one or more parameters associated with the body part of the user on which the device 100 is being worn. Upon sensing of the one or more parameters, the one or more sensors 112 are configured to generate one or more signals indicative of the one or more parameters associated with the body part on which the device 100 is worn.

In an exemplary embodiment, the one or more sensors 112 can include an accelerometer 112-1, a galvanic skin response sensor (GSR) 112-2, a source of electromagnetic radiation 112-3, a photodetector 112-4, and a microphone 112-5.

In another embodiment, the device 100 can include a power storage device 114 configured for supplying power for the operation of the device 100. The device 100 can also be provided with pins or contacts 116 for the charging of the device 100.

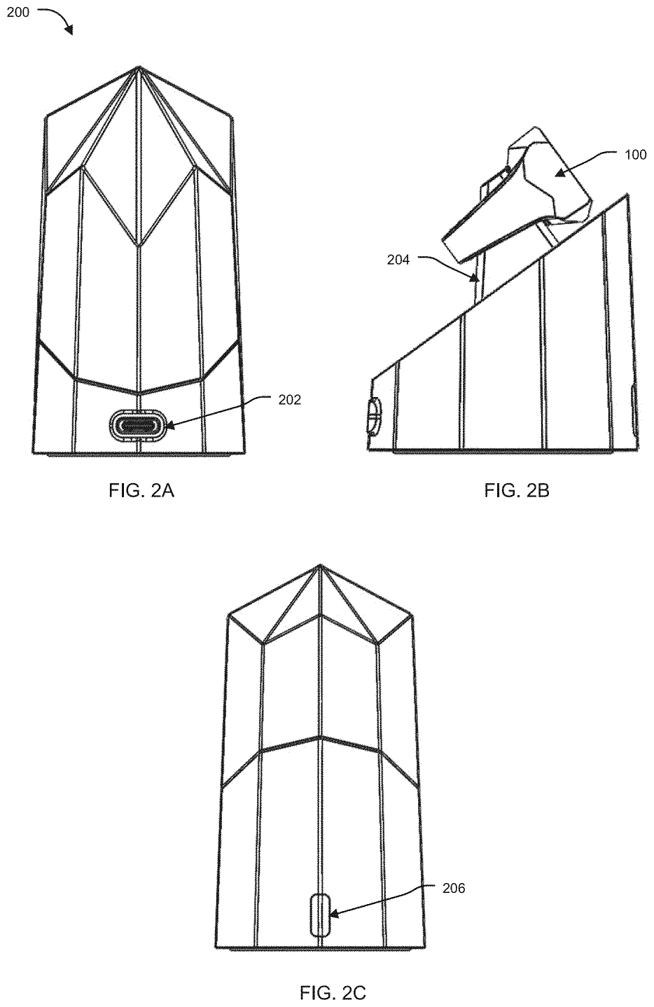

FIGS. 2A-2C illustrate exemplary representations of a charger for the proposed wearable device, in accordance with an embodiment of the present disclosure. The charger 200 can include an input port 202 (ref. FIG. 2A) which can be electrically coupled with a power source through a means such as a cable. The charger 200 can include a mount 204 that is adapted to receive the device 100 such that the contacts 116 of the device is electrically coupled with the mount 204 (ref. FIG. 2B). when power is supplied to the charger 200, in turn, the power is supplied, through the charger 200, to the device 100 to charge the power storage device 114 of the device 100. The mount 204 can couple with the device 100 magnetically so that the device is secured to the mount 204 during charging of the device 100. The charger 200 can include an indicator 206 such as an LED indicator (ref. FIG. 2C) that can indicate the status of charge of the device 100. The indicator 206 can change color of light to indicate complete charge of the device 100.

Referring again to FIGS. 1A-1C, on a top portion of the device 100, is provided a display device 118. The display device 118 can include a light source such as an LED, which can be configured to emit lights of different colors. The display device 118 can also include a light diffuser to soften the emitted light from the light source. The display device 118 can be adapted to emit light of different colors.

In another embodiment, the device 100 can include a processor 120 that is operatively coupled to the sensors 112. The processor 120 is configured to analyze the one or more signals from the one or more sensors 112, and extract one or more attributes from the one or more signals, the one or more attributes being indicative of emotional states of the user.

In another embodiment, the processor 120 can be operatively coupled to a database, which can store a one or more preferred emotional states for the user. The database can further include a set of recommended emotional states corresponding with emotional states of the user, where upon the user adopting the recommended emotional state, the current emotional states of the user tends towards the preferred emotional state.

In another embodiment, the display device 118 is operatively coupled with the processor 120 and is configured to display an indication pertaining to any or a combination of the emotional states of the user and the recommended emotional states for the user.

In another embodiment, the device 100 can include a transceiver 122, which is configured to communicatively couple with an external mobile device. The transceiver 122 can be configured to transmit the first set of signals to the external mobile device through a network. The external mobile device can include a smart phone, a tablet, a cloud-based server, a computer, but not limited to the likes. In an exemplary embodiment, the wireless communication unit can be a Bluetooth Module (IEEE Standard 802.15.1 or IEEE Standard 802.15.4), a Wi-Fi Module (IEEE Standard 802.11), and an IR Module, but not limited to the likes.

In another embodiment, the device 100 can include a flash memory 124 to store information pertaining to a log of operations of the device 100.

In an exemplary embodiment, the components in the housing 110 can be configured on a flexible printed circuit board.

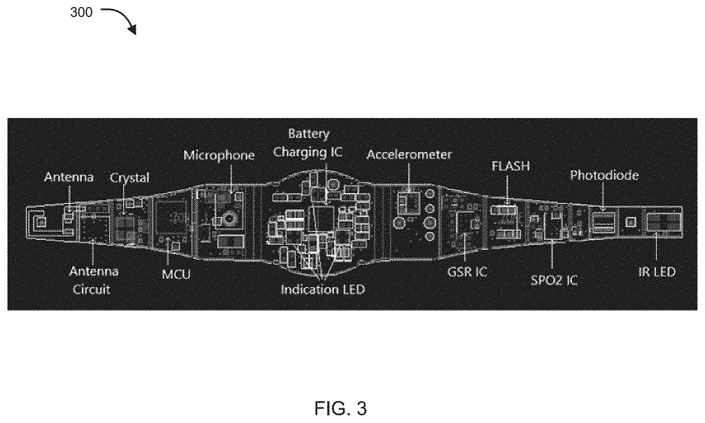

FIG. 3 illustrates an exemplary representation of the flexible printed circuit board of the wearable device. The flexible printed circuit board 300 (PCB) is configured in the housing 110 and offers a platform on which the electrical components of the device 100 are placed.

Referring again to FIGS. 1A-1C, the device 100 is configured to analyze parameters of the body part on which the device 100 is worn, and access emotional states of the user based on the parameters.

In an embodiment, the source of electromagnetic radiation 112-3, and the photodetector 112-4 can constitute a photoplethysmography (PPG) sensor, which can be used in a non-invasive method for detection of cardiovascular pulse and oxygen saturation. The functioning of the PPG sensor can be based on the optical properties of the vascular tissues of the user, based on Beer-Lambert Law. The sources of electromagnetic radiation 112-3 can be operated to emit lights of different wavelengths. The emitted light, after interaction with the blood and tissue of the body part of the user, is detected by the photodetector 112-4. The interactions can be any or a combination of absorption, reflection, and scattering. The intensity of the light detected by the photodetector 112-4 can be measured and the variations caused by blood volume changes are amplified and filtered and recorded as a voltage signal. Light from the sources of electromagnetic radiation 112-3 at two different wavelengths is transmitted through the tissue bed and the photodetector 112-4 measures the unabsorbed light. The flow of blood is heartbeat induced, or pulsatile in nature so the transmitted light changes with time. Red and infrared lights are used for pulse oximetry to estimate the true hemoglobin oxygen saturation of arterial blood.

In another embodiment, the GSR sensor 112-2 can be configured to measure the electrical conductance of a skin of the user. Strong emotion can cause stimulus to sympathetic nervous system, resulting more sweat being secreted by the sweat glands. It can allow identification of strong emotions by attaching two electrodes of the GSR sensor 112-2 in contact with the skin of the user. The GSR sensor 112-2 can utilize the advantage of the electrical properties of the skin of the user, as skin resistance varies with sweat gland activity. The GSR sensor 112-2 can include two electrodes to apply a constant voltage (usually 0.5 V) to the skin. A circuit of the GSR sensor 112-2 contains a very small resistance compared to the skin resistance that is in series with the voltage supplier and the electrodes. The circuit facilitates measuring the skin conductance and its variation by applying Ohm's law (Voltage=Intensity×Resistance=Intensity/Conductance). As the voltage (V) is kept constant, skin conductance (C) can be calculated by measuring the current (I) flow through the electrodes, thereby extracting the one or more bio-physiological parameters associated with electro-dermal activity and skin conductance of the user. In an exemplary embodiment, the one or more bio-physiological parameters associated with the electro dermal activity and skin conductance of the user can be any or a combination of emotional engagement, mental effort, excitement, happiness, fear, shock and arousal, but not limited to the likes.

In an embodiment, the microphone 112-5, along with a flash memory 124 can dynamically record the voice of the user to analyze and extract one or more attributes associated with a voice of the user or persons physically proximal to the user. These attributes can be any or a combination of pitch, sound pressure level, timbre, and time gap between consecutive words of speech. The tonal quality of the human voice changes while expressing various emotions. Detection of human emotions through voice and speech-pattern analysis can prove to be beneficial in improving emotional states recognition ability of the device 100.

In an embodiment, the one or more sensors can be an accelerometer 112-1 to track the motion of the user and hand movement of the user. Humans respond differently to different emotions. This response can include parameters such as abrupt hand movements in case of anger and anxiety, sudden fall, or concussion in case of a mental shock. The accelerometer 112-1 can be configured to monitor these parameters associated with hand movement of user and transfer these parameters in form of the first set of signals to the processing unit to enhance emotional states recognition ability of the wearable computing device 100.

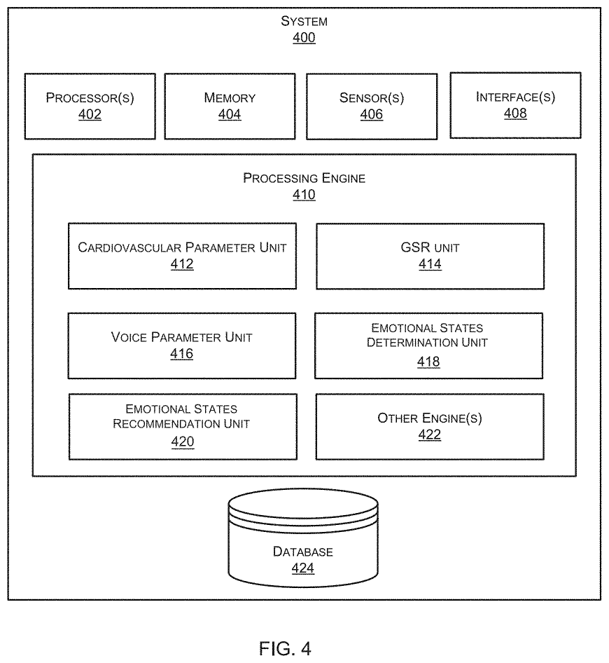

FIG. 4 illustrates an exemplary block diagram for a system implementable on the proposed wearable device to determine emotional states of the user, in accordance with an embodiment of the present disclosure.

It may be appreciated that the embodiments described herein relate to the functions of the sensors (112-1, 112-2 . . . 112-5) in order to assess the emotional states of the user. The device 100 can be configured to include further sensors or can be adapted to have other functions for the existing sensors to extract additional parameters pertaining to the body part of the user on which the device 100 is worn. The present disclosure pertains to an exemplary embodiment of application of the device 100 and persons skilled in the art would appreciate that other related functions of the device as enumerated above can also be included within the scope of this application.

As illustrated, the system 400 can include processor(s) 402. The processor(s) 402 can be implemented as one or more microprocessors, microcomputers, microcontrollers, digital signal processors, central processing units, logic circuitries, and/or any devices that manipulate data based on operational instructions. Among other capabilities, the one or more processor(s) 402 can be configured to fetch and execute computer-readable program code stored in a memory 404 of the system 400. The memory 404 can store one or more computer-readable program code or routines, which may be fetched and executed to create or share the data units over a network service. The memory 404 can include any non-transitory storage device including, for example, volatile memory such as RAM, or non-volatile memory such as EPROM, flash memory, and the like.

The system 400 can also include an interface(s) 408. The interface(s) 408 can include a variety of interfaces, for example, interfaces for database 424 input and output devices, referred to as I/O devices, storage devices, and the like. The interface(s) 408 may facilitate communication of the system 400 with various devices coupled to the system 400. The interface(s) 408 can also provide a communication pathway for one or more components of the system 400. Examples of such components include, but are not limited to, processing engine(s) 410 and database 424.

The processing engine(s) 410 can be implemented as a combination of hardware and programming (for example, programmable instructions) to implement one or more functionalities of the processing engine(s) 410. In examples described herein, such combinations of hardware and programming may be implemented in several different ways. For example, the programming for the processing engine(s) 410 can be processor executable instructions stored on a non-transitory machine-readable storage medium and the hardware for the processing engine(s) 410 include a processing resource (for example, processors) 402, to execute such instructions. In the present examples, the machine-readable storage medium may store instructions that, when executed by the processing resource, implement the processing engine(s) 410. In such examples, the system 400 can include the machine-readable storage medium storing the instructions and the processing resource to execute the instructions, or the machine-readable storage medium may be separate but accessible to system 400 and the processing resource. In other examples, the processing engine(s) 410 can be implemented by electronic circuitry. The database 424 can include database 424 that is either stored or generated as a result of functionalities implemented by any of the components of the processing engine(s) 410.

In an embodiment, the processing engine(s) 410 can include a cardiovascular parameters unit 412, a galvanic skin response unit 414, a voice parameters unit 416, an emotional states determination unit 418, an emotional states recommendation unit 420, and other engine(s) 422. The other engine(s) 422 can implement functionalities that supplement applications or functions performed by the system 400 or the processing engine 410.

In an embodiment, the cardiovascular parameters unit 412 of the proposed system 400 can extract one or more attributes associated with the cardiovascular activity of the user, using the source of electromagnetic radiation 112-3 and the photodetector 112-4. The one or more attributes associated with the cardiovascular activity of the user can be any or a combination of blood oxygen saturation and heartbeat, but not limited to the likes. In an exemplary embodiment, the source of electromagnetic radiation 112-3 and the photodetector 112-4 can be a Photo-plethysmography (PPG) sensor to sense the one or more attributes associated with the cardiovascular activity of the user.

In another embodiment, the galvanic skin response unit 414 of the proposed system 400 can extract one or more attributes associated with the electro-dermal activity and skin conductance of the user, using the GSR sensor 112-2. The one or more attributes associated with the electro-dermal activity and skin conductance of the user can be any or a combination of emotional engagement, mental effort, excitement, happiness, fear, shock, and arousal, but not limited to the likes.

In another embodiment, the emotional states determination unit voice parameters unit 416 of the proposed system 400 can extract one or more attributes associated with the voice of the user, using the microphone 112-5. The one or more attributes associated with the voice of the user can be any or a combination of combination of pitch, sound pressure level, timbre, and time gap between consecutive words of speech, but not limited to the likes.

In an embodiment, the emotional states determination unit 418 of the proposed system 400 can facilitate the processors(s) 402 to compare the extracted one or more attributes and compare these attributes with predetermined attributes associated with existing emotional states. The emotional states determination unit 418 can determine the emotional states associated with the user when at least one of the extracted one or more attributes matches a corresponding predetermined attribute.

In another embodiment, a machine learning based processing can be used to the extract one or more bio-physiological attribute and compare these attributes with the predetermined attribute of existing emotional states and determine the emotional states of the user. These neural network based processing can also facilitate the processing engine 410 to train the emotional states determination unit 418 of the proposed system 400 using the previously determined emotional states of the user, and the attribute associated with it to enhance the accuracy of the neural network.

In an exemplary embodiment, the machine learning based processing can be trained on a historical set of data pertaining to the one or more attributes of the user and the corresponding emotional states of the user.

In another exemplary embodiment, the machine learning based processing can be trained on a historical set of data pertaining to the one or more attributes and corresponding emotional states of the one or more second users.

In another exemplary embodiment, the machine learning based processing can be implemented on the memory 404, and the machine learning based processing can be configured with a classifier model, a generalized regression, and a non-linear regression. The classifier model can further include any or a combination of a support vector machine (SVM), a Gaussian Mixture model (GMM), a k-nearest neighbor classifier and a neural network classifier.

In another embodiment, the emotional states recommendation unit 420 includes a dataset comprising one or more preferred emotional states for the user. The emotional states recommendation unit 420 further includes recommended emotional states corresponding to emotional states of the user, where, upon the user adopting the recommended emotional state, the emotional states of the user tends towards the preferred emotional state.

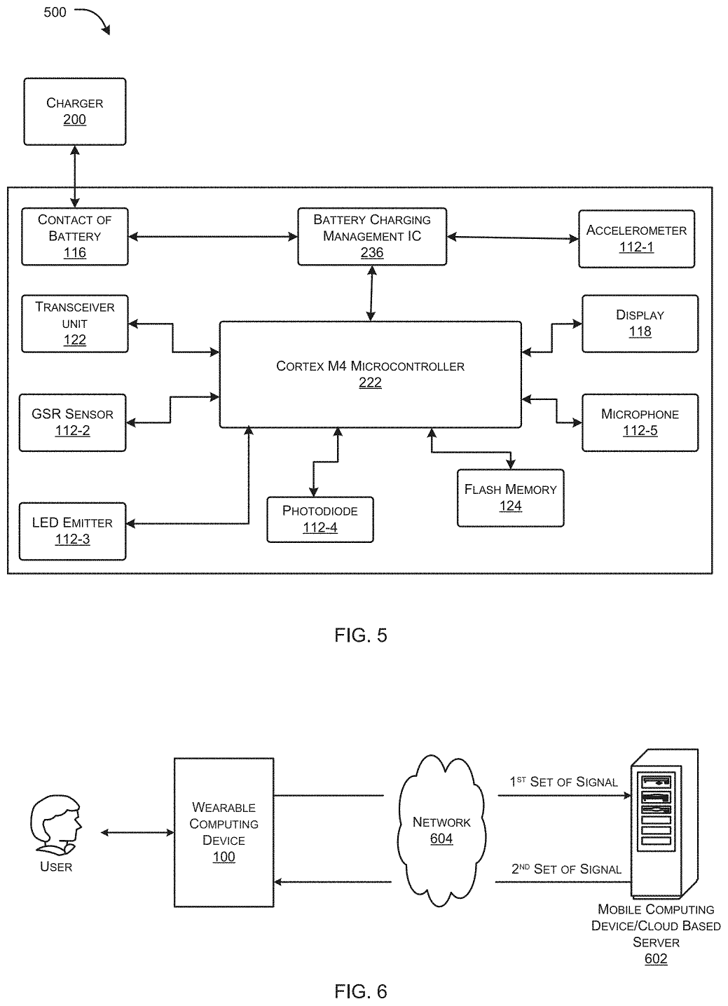

FIG. 5 illustrates exemplary system architecture of the proposed wearable device, in accordance with an embodiment of the present disclosure.

As illustrated, in an embodiment, the exemplary system architecture 500 of the proposed wearable computing device can include a processing unit 120 such as but not limited to a Cortex M-4 Microcontroller 120 and one or more sensors operatively coupled to the processing unit 120. The one or more sensors can include an accelerometer 112-1, a galvanic skin response sensor (GSR) 112-2, a source of electromagnetic radiation 112-3, a photodetector 112-4, and a microphone 112-5. The source of electromagnetic radiation 112-3 and the photodetector 112-4 can be configured to sense one or more attributes associated with cardiovascular activity of the user. The GSR sensor 112-2 can be configured to sense one or more attributes associated with electro-dermal activity and skin conductance of the user. The microphone 112-5 along with a flash memory 124 can be configured to sense one or more attributes associated with voice of the user. The accelerometer 112-1 can be configured to sense one or more attributes associated with hand movement of the user. The one or more sensors can be configured to generate a first set of signals based on the sensed one or more attributes associated with the emotional states of the user.

In an embodiment, the processing unit 120 can be operatively coupled to a transceiver 122, which can be configured to transmit the first set of signals to one or more computing device using the transceiver 122. The transceiver 122 can include a Wi-Fi Module (IEEE Standard 802.11), a Bluetooth (IEEE Standard 802.15.1 or IEEE Standard 802.15.4), but not limited to the likes.

In an embodiment, the system architecture 500 can include a battery charging management IC 502 and a plurality of battery contacts 116. The battery charging management IC 502 can provide linear charging and regulated output to the one or more batteries 114. The battery charging management IC 502 can include a load switch, a manual reset pin with timer and a battery voltage regulator. The plurality of battery contacts 116 can be configured to be electrically coupled the batteries 114 of the device 100 to a battery charger 200.

FIG. 6 illustrates exemplary network architecture in which or with which proposed system can be implemented in accordance with an embodiment of the present disclosure.

As illustrated, in an embodiment, the device 100 worn by the user can sense the one or more attributes associated with the emotional states of the user and generate a first set of signals. The first set of signals can be transmitted to a mobile computing device 602 or cloud-based server 602 through a network 604 using the transceiver 122 of the device 100. The mobile computing device 602 or the cloud-based server 602 can further process the first set of signals and determine the emotional states of the user. The mobile computing device 602 or the cloud-based server 602 can then generate a second set of signals corresponding to the determined emotional set of the user and sends the second set of signals to the wearable computing device 100 through the network 604. Further, the wearable computing device 100 can process the received second set of signals and convert it into a format being displayable on the display of the wearable computing device 100.

The network architecture in which or with which proposed system and wearable device can be implemented is described demonstrating a single user and a single wearable device 100. It is to be appreciated that the present disclosure is not limited to single user or a single wearable device being connected to the server. However, a plurality of users and their corresponding wearable computing devices can be communicatively coupled to the server, and the server can be configured to determine the emotional states for each user.

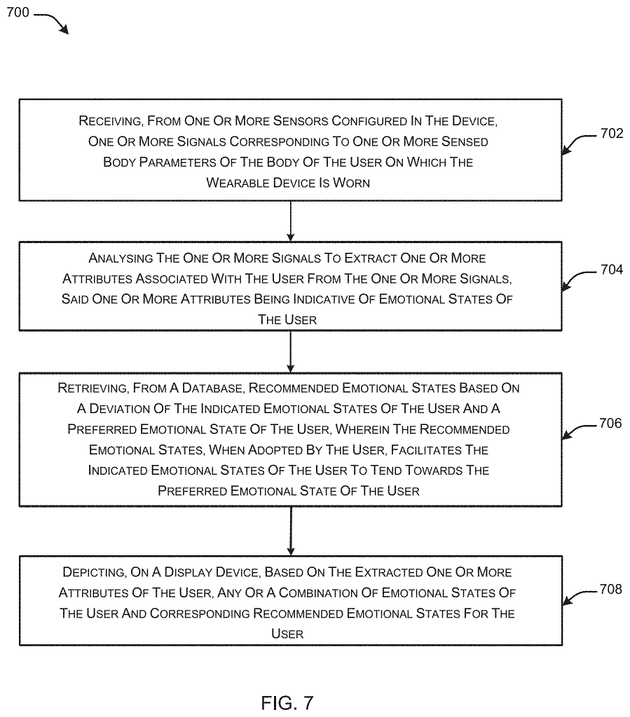

FIG. 7 illustrates an exemplary flow diagram for a method implementable on the proposed wearable device to determine emotional states of the user, in accordance with an embodiment of the present disclosure. The method 700 includes,
702—receiving, from one or more sensors configured in the device, one or more signals corresponding to one or more sensed body parameters of the body of the user on which the wearable device is worn;
704—analyzing the one or more signals to extract one or more attributes associated with the user from the one or more signals, said one or more attributes being indicative of emotional states of the user;
706—retrieving, from a database, recommended emotional states based on a deviation of the indicated emotional states of the user and a preferred emotional state of the user, wherein the recommended emotional states, when adopted by the user, facilitates the indicated emotional states of the user to tend towards the preferred emotional state of the user; and
708—depicting, on a display device, based on the extracted one or more attributes of the user, any, or a combination of emotional states of the user and corresponding recommended emotional states for the user.

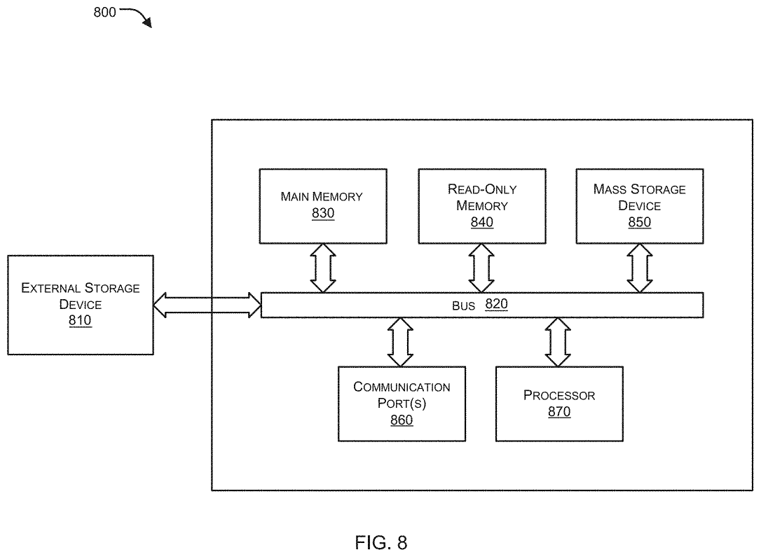

FIG. 8 illustrates an exemplary computer system in which or with which embodiments of the present invention can be utilized in accordance with embodiments of the present disclosure.

As shown in FIG. 8, computer system includes an external storage device 810, a bus 820, a main memory 830, a read only memory 840, a mass storage device 850, communication port 860, and a processor 870. A person skilled in the art will appreciate that computer system may include more than one processor and communication ports. Examples of processor 870 include, but are not limited to, an Intel® Itanium® or Itanium 2 processor(s), or AMD® Opteron® or Athlon MP® processor(s), Motorola® lines of processors, FortiSOC™ system on a chip processors or other future processors. Processor 870 may include various modules associated with embodiments of the present invention. Communication port 860 can be any of an RS-232 port for use with a modem-based dialup connection, a 10/100 Ethernet port, a Gigabit or 10 Gigabit port using copper or fiber, a serial port, a parallel port, or other existing or future ports. Communication port 860 may be chosen depending on a network, such a Local Area Network (LAN), Wide Area Network (WAN), or any network to which computer system connects.

Memory 830 can be Random Access Memory (RAM), or any other dynamic storage device commonly known in the art. Read only memory 840 can be any static storage device(s) e.g., but not limited to, a Programmable Read Only Memory (PROM) chips for storing static information e.g., start-up or BIOS instructions for processor 870. Mass storage 850 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 6102 family) or Hitachi (e.g., the Hitachi Deskstar 6K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc.

Bus 820 communicatively couples processor(s) 870 with the other memory, storage, and communication blocks. Bus 820 can be, e.g. a Peripheral Component Interconnect (PCI)/PCI Extended (PCI-X) bus, Small Computer System Interface (SCSI), USB or the like, for connecting expansion cards, drives and other subsystems as well as other buses, such a front side bus (FSB), which connects processor 870 to software system.

Optionally, operator and administrative interfaces, e.g. a display, keyboard, and a cursor control device, may also be coupled to bus 820 to support direct operator interaction with computer system. Other operator and administrative interfaces can be provided through network connections connected through communication port 860. External storage device 810 can be any kind of external hard-drives, floppy drives, IOMEGA® Zip Drives, Compact Disc-Read Only Memory (CD-ROM), Compact Disc-Re-Writable (CD-RW), Digital Video Disk-Read Only Memory (DVD-ROM). Components described above are meant only to exemplify various possibilities. In no way should the aforementioned exemplary computer system limit the scope of the present disclosure.

It should be apparent to those skilled in the art that many more modifications besides those already described are possible without departing from the inventive concepts herein. The inventive patent matter, therefore, is not to be restricted except in the spirit of the appended claims. Moreover, in interpreting both the specification and the claims, all terms should be interpreted in the broadest possible manner consistent with the context. In particular, the terms “includes” and “including” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc. The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Disclosure

The present disclosure provides a wearable device for a user.

The present disclosure provides a wearable device for a user to determine emotional states of a user.

The present disclosure provides a wearable device which visually depicts emotional states of a user.

1.-27. (canceled)

28. A wearable device for determining emotional states of a user, the wearable device comprising:
a frame adapted to be worn on a body part of the user, the frame comprising:
one or more sensors configured in a housing and operative to sense one or more parameters associated with the body of the user to generate one or more signals indicative of the one or more sensed parameters when the wearable device is worn;
a processor configured in the housing and operatively coupled with the one or more sensors, said processor configured to:
analyze the one or more signals to extract one or more attributes associated with the user from the one or more signals, wherein said one or more attributes being indicative of emotional states of the user; and
retrieve, from a database operatively coupled to the processor, recommended emotional states based on a deviation of the indicated emotional states of the user and a preferred emotional state of the user, wherein the recommended emotional states, when adopted by the user, facilitates the indicated emotional states of the user to tend towards the preferred emotional state of the user; and
a display device configured at a portion of the housing and operatively coupled with the processor, wherein the display device is configured to indicate, based on the extracted one or more attributes of the user, any or a combination of the emotional states of the user and the corresponding recommended emotional states for the user.

29. The device as claimed in claim 28, wherein the device is configured with a power storage device configured to supply power to the one or more sensors, the processor, and the display device, and wherein the device is configured with a set of contacts adapted to receive an external power supply to charge the power storage device.

30. The device as claimed in claim 28, wherein the one or more sensors is the accelerometer, and wherein the processor is configured to:
receive, from the accelerometer, a set of signals associated with movement of the body part of the user,
wherein the processor is configured to analyze the received one or more signals to extract one or more attributes associated with the body part of the user from the received one or more signals,
wherein the one or more attributes are at least partially indicative of the emotional states of the user.

31. The device as claimed in claim 1, wherein the one or more sensors is a combination of the one or more sources of electromagnetic radiation and the photodetector, and wherein the processor is configured to:
operate the one or more sources of electromagnetic radiation to emit electromagnetic radiation corresponding to one or more wavelengths, the electromagnetic radiation aimed towards the body part of the user; and
receive, from the photodetector, one or more signals associated with interaction of the emitted electromagnetic radiation with the body part of the user, the interaction being any or a combination of scattering, reflection, and absorption, wherein the processor is configured to analyze the received one or more signals to extract one or more attributes associated with the body part of the user from the received one or more signals, said one or more attributes being indicative of any or a combination of pulse rate and oxygen saturation of the user, and
wherein the one or more attributes are at least partially indicative of the emotional states of the user.

32. The device as claimed in claim 28, wherein the one or more sensors is the galvanic skin response sensor, and wherein the processor is configured to:
receive, from the galvanic skin response sensor, one or more signals associated with electrical conductivity of the body part of the user,
wherein the processor is configured to analyze the received one or more signals to extract one or more attributes associated with the body part of the user from the received one or more signals, said one or more attributes being indicative of quantity of sweat present on the body part of the user and temperature of the body part of the user, and
wherein the one or more attributes are at least partially indicative of the emotional states of the user.

33. The device as claimed in claim 28, wherein the one or more sensors is the microphone, and wherein the processor is configured to:
receive, from the microphone a one or more signals associated with speech of the user,
wherein the processor is configured to analyze the received one or more signals to extract one or more attributes associated with the body part of the user from the received one or more signals, said one or more attributes pertaining to any or a combination of pitch, timbre, volume, speed and tone, and
wherein the one or more attributes are at least partially indicative of the emotional states of the user.

34. The device as claimed in claim 28, wherein the processor is configured to retrieve the recommended emotional states for the user from a database operatively coupled to it.

35. The device as claimed in claim 28, wherein the display device is configured to emit a first light of a predefined color out of a set of colors, said predefined color being indicative of the emotional states of the user, and wherein the predefined color is selected based on a predefined association of the set of colors with different emotional states of the user.

36. The device as claimed in claim 28, wherein the display device is configured to emit a second light of a predefined color out of a set of colors, said predefined color being indicative of the recommended emotional states for the user, and wherein the predefined color is selected based on a predefined association of the set of colors with different emotional states of the user.

37. The device as claimed in claim 28, wherein the display device comprises a light source coupled with a diffuser, the light source adapted to emit light of different colors and the diffuser adapted to provide a soft illumination of the display device.

38. The device as claimed in claim 28, wherein the device comprises a memory unit configured to store information pertaining to a log of operations of the device, said log of operations comprising any or a combination of the one or more signals from the one or more sensors, the extracted one or more attributes, the emotional states of the user, the corresponding retrieved recommended emotional states for the user, and a time stamp for the log of operations.

39. The device as claimed in claim 28, wherein the device comprises a transceiver unit operatively coupled with the processor, the transceiver unit configured to communicatively couple the device with an external mobile device, the communicative coupling being a wireless communicative coupling through any or a combination of Wi-Fi, Bluetooth, radio, and mobile network.

40. The device as claimed in claim 39, wherein the external mobile device is configured with an application executable to communicatively couple with the wearable device.

41. The device as claimed in claim 39, wherein the external mobile device is a remote server, the server operatively coupled with a computing device, said computing device comprising a processor coupled with a memory, said memory storing instructions executable by the processor to perform one or more analyses of the log of operations of the device.

42. A system to determine emotional states of a user using a wearable device of claim 1, the system comprising:
a processor operatively coupled with a memory, the memory storing instructions executable by the processor to:
receive, from one or more sensors configured in the device operatively coupled to it, one or more signals corresponding to one or more sensed body parameters of the body of the user on which the wearable device is worn, wherein the processor is operative to analyze the one or more signals to extract one or more attributes associated with the user from the one or more signals, said one or more attributes being indicative of emotional states of the user;
retrieve, from a database operatively coupled to the processor, recommended emotional states based on a deviation of the indicated emotional states of the user from a preferred emotional state of the user, wherein the recommended emotional states, when adopted by the user, facilitates the indicated emotional states of the user to tend towards the preferred emotional state of the user; and
depict, on a display device operatively coupled to the processor, based on the extracted one or more attributes of the user, any or a combination of emotional states of the user and the corresponding recommended emotional states for the user.

43. The system as claimed in claim 42, wherein the processor is operatively coupled with a learning engine, the learning engine configured to determine the emotional states of the user from one or more analyses performed on the extracted one or more attributes of the user.

44. The system as claimed in claim 43, wherein the learning engine is trained to determine the emotional states of the user based on historical data comprising the one or more attributes and the corresponding emotional states of the user.

45. The system as claimed in claim 43, wherein the learning engine is trained to determine the emotional states of the user based on simulated historical data comprising one or more attributes and the corresponding emotional states.

46. The system as claimed in claim 42, wherein said system comprises one or more devices of claim 1 associated with any of one or more users, the system configured to depict any or a combination of emotional states of any of the one or more users and corresponding recommended emotional states for any of the one or more users.

47. A method to determine emotional states of a user using the wearable device of claim 1, the method comprising:
receiving, at a processor from one or more sensors configured in the device operatively coupled to it, one or more signals corresponding to one or more sensed body parameters of the body of the user on which the wearable device is worn,
wherein the processor is operative to analyze the one or more signals to extract one or more attributes associated with the user from the one or more signals, said one or more attributes being indicative of emotional states of the user;
retrieving, at the processor from a database operatively coupled to the processor, recommended emotional states based on a deviation of the indicated emotional states of the user and a preferred emotional state of the user,
wherein the recommended emotional states, when adopted by the user, facilitates the indicated emotional states of the user to tend towards the preferred emotional state of the user; and
depicting, on a display device operatively coupled to the processor, based on the extracted one or more attributes of the user, any, or a combination of the emotional states of the user and the corresponding recommended emotional states for the user.
